(19) United States
(12) Patent Application Publication
(10) Pub. No.: US 2003/0119073 A1
(43) Pub. Date: Jun. 26, 2003

(54) Sensors and methods of detection for proteinase enzymes

(76) Inventors: Stephen Quirk, Alpharetta, GA (US); David John Tyrrell, Appleton, WI (US)

Correspondence Address: JOHN S. PRATT, KILPATRICK STOCKTON LLP (KIMBERLY CLARK), 1100 PEACHTREE STREET, SUITE 2800, ATLANTA, GA, 30309, US

(21) Appl. No.: 10/026,393

(22) Filed: Dec. 21, 2001

(51) Int. Cl. G01N 33/573

(52) U.S. Cl. 435/7.4

(57) ABSTRACT

Sensors for detecting catabolic proteinase enzymes and proenzymes in the fluid of a human or animal and methods for detecting the enzymes and then providing treatment that is specific for the detected enzyme are disclosed. The sensors of the present invention can be used to detect catabolic proteinase enzymes and proenzymes in the fluid of chronic wounds of humans and animals. Upon detection of any proteinase enzyme, the wound can be treated with an inhibiting complex that is specific for the detected enzyme or proenzyme. Enzymes such as matrix metalloproteinases and human neutrophil elastase in the active and proenzyme form can be detected and treatment provided with inhibitors for the detected enzyme.

### FIELD OF INVENTION

The present invention relates to the detection of catabolic proteinase enzymes and proenzymes in the fluid of humans and animals. More particularly, the invention relates to devices and methods for detecting catabolic proteinase enzyme activity and proenzyme presence in the wounds of humans and animals and then providing a treatment that is specific for the proteinase enzyme or proenzyme that was detected.

### BACKGROUND OF THE INVENTION

Effective ways to treat wounds is a major medical concern because many patients develop chronic wounds, causing increased healthcare provider costs. Open cutaneous wounds represent one major category of chronic wounds, which also include burn wounds, neuropathic ulcers, pressure sores, venous stasis ulcers, and diabetic ulcers. In the U.S. alone, the prevalence of chronic wounds has been estimated to occur in nearly 6 million patients. The cost involved in treating these wounds averages $3,000 per patient, totaling over $13 billion per year for healthcare costs in the United States.

Catabolic proteinase enzymes, such as matrix metalloproteinases (MMPs) and human neutrophil elastase (hNE), have been implicated in causing chronic wounds. In normal tissues, cellular connective tissue synthesis is offset by extracellular matrix degradation, with the two opposing effects existing in dynamic equilibrium. Degradation of the matrix is brought about by the action of catabolic proteinase enzymes (proteinase enzymes) released from resident connective tissue cells and invading inflammatory cells. Normally, these catabolic enzymes are tightly regulated at the level of their synthesis and secretion and also at the level of their extracellular activity. Extracellular control occurs primarily by regulation with specific regulatory proteins, such as tissue inhibitors of metalloproteinases, which form complexes with MMPs. These complexes prevent MMP action. Cellular level control of MMP activity occurs by controlling the activation of proenzyme forms in part by down regulating MMP gene expression and by down regulating the expression of the membrane bound MMPs (MT-MMP) that activate the excreted proenzyme form of the MMP.

Chronic wounds that do not heal well are characterized by an increase in the activity of proteinase enzymes including, but not limited to, matrix metalloproteinases (MMPs). These enzymes are responsible for the continued degradation of newly formed basal extracellular matrix (ECM). The stable formation of this matrix marks a committed entry into the healing process; however, constant ECM turnover results in an inability of the chronic wound to heal. There are three MMPs that are particularly problematic in chronic wounds; MMP-1 or interstitial collagenase, MMP-8 or neutrophil collagenase, and MMP-9 or gelatinase B. In addition, another catabolic proteinase enzyme, human neutrophil elastase, is secreted by activated neutrophils and plays a significant role in ECM turnover by directly degrading matrix constituents or by indirectly activating other matrix-degrading enzymes that include MMPs.

Under normal circumstances, MMPs are prevented from destroying the wound bed by the action of four Tissue Inhibitors of MetalloProteinase (TIMPs) that form very specific inhibitory complexes with the MMPs. Each TIMP only inhibits a specific subset of MMPs. In chronic wounds the ratio of MMP to TIMP is high, such that most of the MMPs are uninhibited. In fact, with elevated proteinase levels, the TIMP molecules themselves can be hydrolyzed. No naturally occurring TIMP molecule that singly inhibits all types of MMPs has been found to exist.

There are currently approximately 23 accepted members of the MMP enzyme family, including membrane-bound forms. MMPs include the collagenases, stromelysins, and gelatinases. All of these proteinases are found in the chronic wound microenvironment. MMPs are biosynthetically produced in an inactive proenzyme form. Proteolytic cleavage of the proenzyme that results in MMP activation can be initiated by a separate class of membrane bound MMPs or through enzymes present in the wound fluid that include neutrophil elastase or plasmin. The proenzyme leader sequence is approximately 100 amino acids in length and is found at the extreme amino terminus of the protein. Detection of inactive proMMPs is important for proper chronic wound management, but it has proven to be difficult to accomplish in practice.

Since the level of these enzymes is constantly in flux within a chronic wound, it is therapeutically important to specifically identify which proteinase, whether an enzyme or proenzyme, is at high levels. Many approaches have been suggested to control MMP activity. Levy, Wojtowicz-Praga, and Duivenvoorden have investigated the use of small molecules, while Odake has investigated peptide-based inhibitors, and Su has used anti MMP antibodies. None of these investigators have used rapid detection of catabolic enzymes and proenzymes to treat chronic wounds.

Chronic wounds can be treated effectively by detecting the presence of specific catabolic enzymes and proenzymes. The ability to detect proteinase enzymes fast, accurately, and inexpensively would help to expedite treatment. Rapid detection of catabolic enzymes and proenzymes allows for immediate treatment with an inhibitory agent that is specific for each enzyme that is detected. Current methods of detecting enzymes can be cumbersome and costly and require highly trained technicians. Testing is performed in laboratories and results may take hours or days. Therefore, treatment of chronic wounds is delayed significantly, resulting in greater catabolic activity. The present invention is directed to overcoming these and other deficiencies in the art.

### SUMMARY OF THE INVENTION

The present invention relates to a sensor and methods to detect and identify target proteinase enzymes and proenzymes in the fluid of a human or animal, particularly in the fluid from a wound, and then inhibiting the activity of the enzyme or preventing activation of the proenzyme that was detected. The present invention also relates to the effective treatment of chronic wounds by detecting the presence of catabolic enzymes and proenzymes and then providing a treatment that is specific for the enzyme that was detected.

The present invention relates to a sensor for detecting and identifying proteinase enzymes in a fluid. The sensor comprises a sample reservoir in fluid communication with at least one reaction site and a collection area. A signal element and a target antibody bindable to a specific portion of a target proteinase enzyme or proenzyme are disposed within the sample reservoir. The reaction site contains a capture antibody that is bindable to another portion of the target proteinase enzyme or proenzyme. The capture antibody is stationary within the reaction site. The target antibody and capture antibody recognize different epitopes on the proteinase enzyme. When the target proteinase enzyme is exposed to the target antibody, a complex of target antibody target proteinase enzyme is formed. When this complex is exposed to a capture antibody bindable to the target proteinase, a conjugate of target antibody target proteinase enzyme complex and capture antibody is formed. For purposes of the invention, the target antibody bound to the target proteinase enzyme or proenzyme is referred to as the “complex”, and the target antibody target proteinase enzyme complex bound to the capture antibody is referred to as a “conjugate”. The formation of the conjugate indicates the presence of the target proteinase in the reaction site because the capture antibody is stationary in the reaction site and causes a concentrated presence of signal element in the reaction site. This results in a detectable or measurable manifestation of the signal element in the reaction site. Specific catabolic proteinase enzymes can be detected and measured in the fluid of a human or animal by exposing a sample taken from the human or animal to a signal element and a target antibody that is bindable to a target proteinase enzyme to form a complex. The complex is then exposed to a stationary capture antibody that is bindable to the target proteinase enzyme of the complex, forming a conjugate. The concentration of conjugate in the area where the capture antibody is stationary causes a detectable or measurable manifestation of the signal element. The presence of a target enzyme or proenzyme is determined by measuring or viewing the reaction site to determine the presence of the signal element in concentrations greater than the reaction area prior to the sample introduction. Advantageously, the proteinase enzyme can be identified when reaction sites contain known antibodies to only one proteinase enzyme.

Treatment can be provided that is targeted to the proteinase enzymes or proenzymes that are detected and identified in the fluid of a human or animal. For example, when a specific proteinase enzyme or proenzyme is detected in a sample from a chronic wound of a human or animal, a specific inhibitor for the detected target enzyme or proenzyme can be applied to the chronic wound to reduce the catabolic activity of the enzyme and/or activation of the proenzyme which can lead to enhanced healing.

### BRIEF DESCRIPTION OF THE DRAWINGS

FIG. 1A is a schematic representation of one aspect of a proteinase enzyme sensor made in accordance with the present invention.

FIG. 1B is a cross-sectional side view of the proteinase enzyme sensor of FIG. 1A illustrating a sample reservoir, a reaction site, and a collection area.

FIG. 1C is a cross-sectional side view of another aspect of a proteinase enzyme sensor made in accordance with the present invention illustrating a plurality of reaction sites.

FIG. 2 is a schematic representation of an example of an assay for detection of proteinase enzymes.

FIG. 3A is a schematic representation of one aspect of a proteinase enzyme sensor made in accordance with the present invention having a plurality of reaction sites.

FIG. 3B is a cross-sectional side view of a proteinase sensor of FIG. 3A illustrating a plurality of reaction sites.

FIG. 4 is a schematic representation of a proteinase sensor made in accordance with the present invention demonstrating the detection of proteinase enzymes.

FIG. 5 is an example of a graph demonstrating by ELISA assay the relative fluorescence of the antibodies used in the detection of proteinase enzymes.

FIG. 6 is an example of a graph of the results of an ELISA assay showing the range of detection of the antibodies.

FIG. 7 is an example of a graph of an ELISA assay demonstrating that an antibody produced against the activation domain of MMP-9 can detect the pro form of MMP-9.

FIG. 8 is an example of a graph demonstrating surface plasmon resonance detection of antibody binding.

### DETAILED DESCRIPTION OF THE INVENTION

The invention relates to a sensor and methods to detect and identify proteinase enzymes and proenzymes in the fluid of a human or animal, particularly in the fluid from a wound. The invention further relates to methods of treating chronic wounds by detecting and identifying the presence of proteinase enzymes and proenzymes, either singly or simultaneously, and then treating the wound with inhibitors that are specific for the proteinase enzymes and proenzymes found in the wound. For purposes of the present invention, the terms treat and treatment describe inhibiting the active enzymes and preventing the activation of the proenzymes, either singly or concurrently.

For purposes of the invention, the term animal defines any animal subject to chronic wounds, including but not limited to, dogs, cats, birds, horses, caffle, hogs, sheep, goats, zoo animals, and the like.

For purposes of the present invention, the term proteinase enzyme comprises active enzymes, proenzymes and other catabolic enzymes and proenzymes including, but not limited to, hNE, MMP-1, MMP-8, MMP-9, proMMP-1, proMMP-8, or proMMP-9.

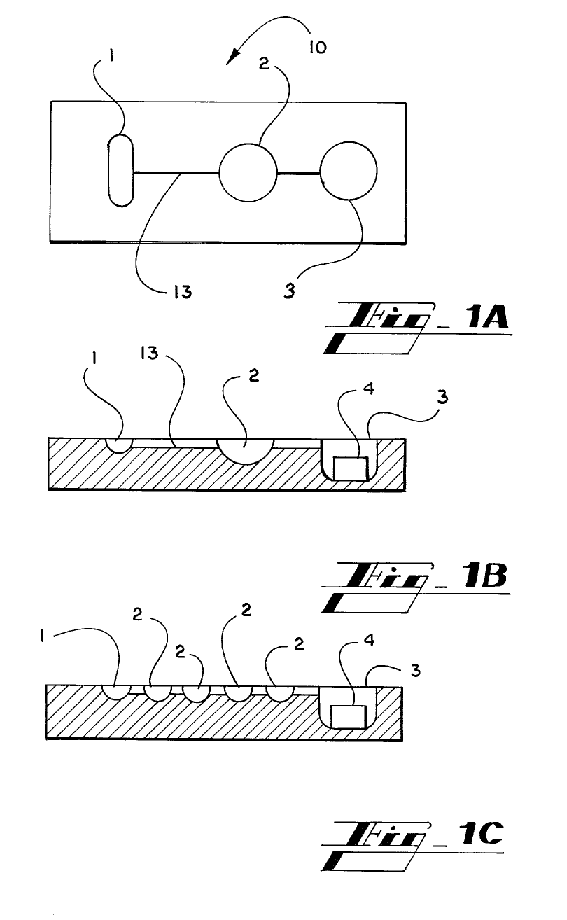

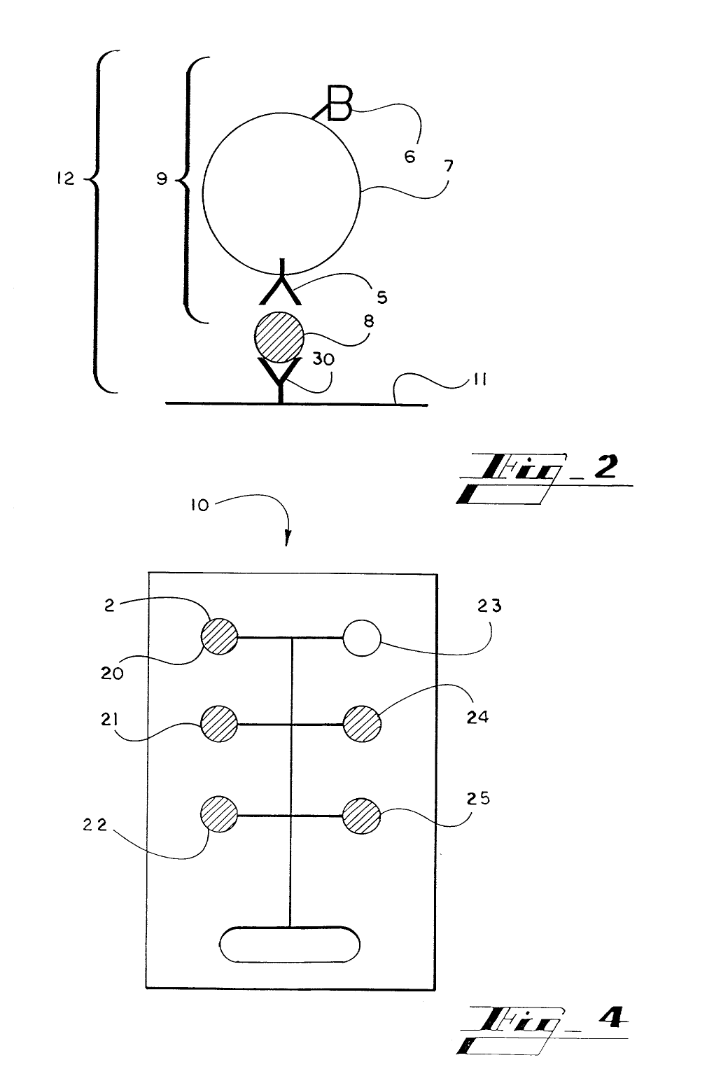

For a fuller understanding of the nature of the invention, reference should be made to the following detailed description taken in connection with the accompanying drawings. Referring to the drawings wherein like reference numerals designate corresponding parts throughout the several Figures, reference is first made to FIGS. 1A, 1B, and 1C, which illustrate a sensor for detecting proteinase enzyme in a fluid of a human or animal. The sensor comprises a sample reservoir (1) in fluid communication (13) with at least one reaction site (2). Alternatively, the sensor further comprises a collection area (3) in fluid communication with the reaction site.

Fluid communication between the sample reservoir and the reaction site can be accomplished by any fluid flow means known in the art such as, but not limited to, channels, capillary tubes, wicks, or any combination thereof. The fluid flow means could represent any geometric configuration including curved, perpendicular, parallel or any combination thereof. Advantageously, any capillaries or channels could comprise any internal geometric configuration including, but not limited to, oval, circular, or having at least one angle, such as triangular. Capillary attraction, wicking, gravity, pressure, and combinations thereof, can affect the flow of fluid from the sample reservoir through the sensor to the collecting area. The most practical and cost effective aspects of the sensor would utilize capillary attraction, wicking, gravity, and combinations thereof.

The purpose of the collection area is to collect the waste or residual fluid from the sample. Waste is everything that is not trapped in the reaction site(s). The collection area can include, but is not limited to, a reservoir, an opening, a flat plane with a slight depression, or any structure that would allow for waste to flow out of the reaction site(s) and into an area for holding. A collection area can further comprise an absorbent pad (4) made from any absorbent material such as cellulose, cotton, latex sponge, a filter, any porous or semi-porous membrane, or any combination of materials.

Sensors of the present invention can comprise a plurality of reaction sites for simultaneous detection of more than one proteinase enzyme as illustrated in FIGS. 1C, 3A and 3B. Advantageously, the reaction sites are in fluid communication with each other and the collection area. The reaction sites can be arranged in series as in FIG. 1C, in parallel, or in any configuration to allow the sample to flow from the sample reservoir through each reaction site, either successively or alternately, and ultimately enter the collection area. The number of reaction sites can total the number of target enzymes that need detecting and can alternatively also contain reaction sites for controls. Advantageously, each reaction site can contain target antibodies to only one proteinase enzyme. Ideally, reaction time is less than about one hour. More advantageously, reaction time is less than about 30 minutes. Most advantageously, reaction time is less than about 10 minutes.

For ease of use and transportability, the sensor (10) can further comprise a housing (15). The configuration of the sensor and housing can include that of any device that moves fluid from one end of a sensor to another, including, but not limited to, lateral flow devices. The housing contains a viewing area (16) for viewing the presence or absence of a detectable or measurable manifestation indicating the presence or absence of the proteinase enzyme. The sensors of the present invention are inexpensive to produce, provide enough sensitivity to detect proteinase enzymes, can be used by non-technical personnel, and are fully disposable.

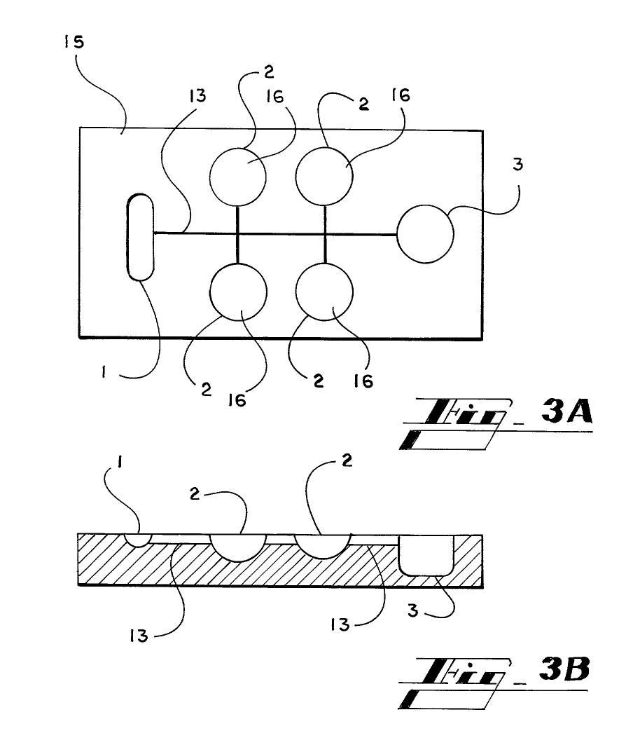

FIG. 2 is a schematic representation of the assay of the sensor. A signal element (6) and a target antibody (5) are attached to a particle (7) and disposed within the sample reservoir. The target antibody is bindable to a target proteinase enzyme (8) upon exposure to a sample of fluid containing target proteinase enzyme. The target antibody forms a complex with the target proteinase enzyme (9). The sample of fluid containing the target antibody proteinase enzyme complex flows to at least one reaction site that is in fluid communication with the sample reservoir and a collection area. Capture antibodies (30) bindable to the target proteinase enzyme of the complex are attached to the surface of the reaction site (11). The target antibodies and capture antibodies are specific for only one target proteinase enzyme, however, the target antibody recognizes a different epitope on the proteinase enzyme from the epitope that the capture antibody recognizes. When the sample containing the complex is exposed to the capture antibodies in the reaction site, conjugate (12) of target antibody target proteinase enzyme complex capture antibody is formed. As the concentration of conjugate increases in the reaction site, a detectable or measurable manifestation of the signal element occurs due to the presence of signal element in the conjugate.

The antibody and the signal element can be coupled to a particle by one of several chemical methods known to those skilled in the art. Examples include, but are not limited to, a method using carbodiimide chemistry to link carboxyl and free amino groups. For example, the particles can have free carboxylates that are activated to a succinimide or maleimide ester. Reaction with a free amino group on the signal element or the antibody results in coupling. Alternatively, linkage could occur via a disulfide exchange reaction or any method known in the art.

The antibodies themselves can be monoclonal antibodies produced from mice and/or hybridoma cell lines, polyclonal antibodies produced in rabbits, sheep, goats, or other animals used to produce antisera, or recombinant antibodies or antibody fragments, including but not limited to F(ab) and F(ab′)2 or single-chain recombinant fragments. Antibodies for use in the present invention are available commercially from Sigma Chemicals. Alternatively, monoclonal or polyclonal antibodies can be made by any method known in the art.

In accordance with the present invention, target antibodies can be disposed within the sample reservoir upon a particle including, but not limited to, latex, polymers, gold, silicon, glass, metal, bacterial or fungal cells, or any particle to which the signal element and target antibody can be attached by any method known in the art. Advantageously, the size of particles would range from about 100 nm to about 1 micron. Such particles can be disposed within an absorbent pad, wick, or sponge, as a pellet, as loose particles within the sample reservoir, or any combination thereof.

The signal element can be any composition containing any indicator known in the art that provides a detectable and/or measurable manifestation, without a chemical reaction, when the signal element is concentrated in one location. Signal elements can include, but are not limited to, colorimetric compounds, fluorophores, chemo-illuminescent compounds, magnetic compounds, radioactive compounds, compounds that can be detected potentiometrically, light diffraction elements, or combinations thereof. A detectable or measurable manifestation of the signal element can be any means of determining the presence of the element, including but not limited to, any visible means of detection, or any means of detection using devices. Devices for detection include, but are not limited to, counters, spectrophotometers, imaging equipment, magnetic detection devices, radio-activity detection devices, light diffraction measurement devices, potentiometric detection devices, or any combination thereof.

Examples of colorimetric compounds that can be used include, but are not limited to, commercially available dyes such as those available from Bangs Laboratories, Inc.

Capture antibodies are stationary and remain within the reaction site after forming the conjugate. The capture antibodies can be retained in the reaction site by chemically cross-linking the antibody to the surface of the reaction site, physically adsorbing the antibody to the surface, or by any means known in the art that would not alter the properties of the antibody.

Alternatively, the presence of catabolic proenzymes can be detected by the formation of a diffraction image as disclosed and described in U.S. Pat. No. 5,922,550, U.S. Pat. No. 6,020,047, U.S. Pat. No. 6,221,579 and International Publication Nos. WO 98/27417 and WO 00/34781, which are herein incorporated by reference in their entirety. The capture antibody can be printed onto a substrate, for example a plastic film, in a defined pattern such that the capture antibody-printed film does not diffract electromagnetic radiation when the electromagnetic radiation is reflected off of or transmitted through the capture antibody-printed film but diffracts electromagnetic radiation after the capture antibody-printed film is exposed to the target proteinase enzyme and the enzyme has bound, reacted or otherwise associated with the capture antibody. Thus, the presence of proteinase enzyme can be determined by a measurable change in diffraction of light that is transmitted through, or reflected off of the substrate surface. If light or other electromagnetic radiation is to be transmitted through the surface of a substrate to detect diffraction, it is desirable that the substrate is transparent or at least partially transparent to the light or other electromagnetic radiation that will be used to detect diffraction.

In addition, as described in the aforesaid references, particles may be used as a signal element with the present invention and can be diffraction enhancing materials including, but not limited to, glass, cellulose, synthetic polymers or plastics, latex, polystyrene, polycarbonate, bacterial or fungal cells, or any combination thereof. For detection of diffraction, a desirable particle ranges in size from about 0.05 micrometers to about 100.0 micrometers in diameter. The composition, structural and spatial configuration of the particle is not critical to the present invention, however, it is desirable that the difference in refractive index between the substrate and the particle is between about 0.1 and about 1.0.

In one aspect of the present invention, the presence of proteinase enzymes in the fluid of humans and animals can be detected using the sensors of the present invention. Advantageously, the presence of proteinase enzymes in the fluid of chronic wounds of humans and animals can be detected. In one aspect of the invention, a method for detecting the presence of at least one proteinase enzyme in the fluid of a human or animal comprises providing a sample of the fluid and exposing the sample to a signal element and at least one target antibody that is bindable to a target proteinase enzyme to form a target antibody target proteinase enzyme complex. The complex is then exposed to a capture antibody bindable to the target proteinase enzyme in the complex to form a conjugate. The capture antibody is attached to the surface of the reaction site and only complexes that are bound to the capture antibody will be retained in the reaction site. Ideally, capture antibodies for one target proteinase enzyme are placed in each reaction site. As the concentration of conjugate increases in the reaction site, it causes a detectable or measurable manifestation due to the concentration of the signal element present in the complex. Any excess sample that contains unconjugated fluid flows to a collection area. The identity of the detected target proteinase enzyme can be determined by noting a presence or absence of the detectable or measurable manifestation in the viewing area. The sensor of the present invention can detect active enzymes or proenzymes including, but not limited to MMP-1, MMP-8, MMP-9, hNE, pro MMP-1, pro MMP-8, proMMP-9, or any combination thereof. Enzymes can be detected singly or more than one can be detected simultaneously.

In another aspect of the invention, a sample of fluid is removed with a small pipette from the chronic wound of a human or animal. The sample is then added to the sample chamber of a sensor as shown in FIG. 4, which contains polystyrene beads coated with target antibodies and a dye. If a proteinase enzyme is present in the chronic wound fluid, it will bind to the target antibody that is bindable to the target enzyme and form a target antibody proteinase enzyme complex. The sample containing the complex flows to the first reaction site and location of capture antibodies. Each reaction site has different capture antibodies bindable to only one proteinase enzyme. Capture antibodies bindable to the proteinase enzyme present in the complex bind the complex to form a conjugate. Conjugates are held in the reaction site and any complexes that did not form conjugate flow to the next reaction site where the same process takes place. Alternatively, sample could flow down one fluid communication means and aliquots of the sample could flow to individual reaction sites positioned along the fluid communication means. Once the fluid has passed through all reaction sites, any remaining sample flows to a collection area. Each reaction site contains capture antibodies known to bind to a specific target proteinase enzyme. The conjugate formed in each reaction site results in an increasing concentration of beads containing the dye molecule. The concentration of beads held by the conjugate causes a detectable or measurable manifestation of the signal element, such as the presence of a color. Alternatively, the signal element could be a fluorophore, potentiometric element or radioactive element that is measured by a device for detection. Any reaction sites with color indicate the presence of an enzyme. Any sites without color indicate that the enzyme was not present. In FIG. 4, the presence of color in sites 20, 21, 22, 24, and 25 indicate the presence of MMPs 1, 8, 9, and pro MMP1 respectively. The absence of color in 23 indicates that no hNE was detected. Advantageously, reaction sites for positive and negative controls can be provided.

In another aspect of the invention, a sample of chronic wound fluid is removed from the wound of a human or animal with a small pipette and added to the sample chamber. Sample can flow in series from one reaction site to the next. Alternatively, the sample can flow along one channel and aliquots of sample can be directed individually to reaction sites that are positioned perpendicular to the flow channel. The fluid is moved from the sample chamber to four separate antibody reaction sites and into a collection area by the fluid flow caused by a wick in the collection area. Each reaction area contains the reacting capture antibody at a fixed and known concentration. If the specific proteinase is present in the chronic wound fluid, it will bind to the antibody and form a specific complex. If this complex forms, it will be detected by the user via a developing color change. Alternatively signal elements could include fluorescence or chemoilluminescence. If the proteinase concentration is greater than the capture antibody concentration at the first reaction site, it will continue to be moved forward to the second reaction site. A colored complex will then be concentrated at the second reaction site. If the proteinase concentration is equal to the sum of the captured antibody concentrations on reaction sites one and two, then the test is finished. Otherwise proteinase will continue to move to reaction sites three and four. The number of reaction sites that develop a color change will indicate the approximate proteinase concentration. The concentration of the capture antibody at each reaction site will be determined so that the entire assay spans the proteinase levels found in chronic wounds. The concentration of capture antibody can be in excess so that the single reaction site would not be saturated. Concentrations can be indicated for example, by markings on a housing containing the sensor, or alternatively, by comparing the color of the reaction site with the color of known concentrations of the conjugate. When the color of the test result is matched to the color of the known concentration, the amount of enzyme can be determined. The known concentration colors can be provided, for example, on a chart.

Methods of detecting proteinase enzymes according to the present invention can include obtaining fluid from the actual wound, from fluid that has been obtained from the human or animal, or from the bandage material that has been removed from a human or animal. The test can be performed while at the patient's bedside, or alternatively, the sample can be transported to another location for testing. One proteinase enzyme or several can be detected simultaneously. One advantageous aspect of the method of the present invention is the ability to simultaneously detect proteinase proenzymes, as well as activated forms of the enzyme. As the body produces proteinase enzymes such as MMPs, it does so in an inactive form. To activate the proenzyme, a proteolytic cleavage event cleaves the enzyme prodomain, or approximately the first 100 amino acids. The resulting Cter domain, or carboxy terminus of the protein, becomes the mature and active form of the proteinase. The presence of the proenzymes in the wound contributes to the catabolic activity as they become activated. The sensor of the present invention makes it possible to simultaneously detect the presence of MMPs-1,8,9 and hNE, as well as the proenzyme forms. As a result, the proenzymes can be treated to prevent activation. This is an advantage in picking the correct anti-protease therapy and in judging the overall health of the wound.

The present invention relates to a method of treating chronic wounds by detecting the presence of catabolic proteinase enzymes and proenzymes, and then treating with inhibitors that are specific for the proteinase enzymes and proenzymes that are detected. It is preferable to detect and treat both the active enzyme and proenzyme form of catabolic enzymes in chronic wounds because proenzymes are continually activated by other MMP complexes or other enzymes that include plasmin to form active enzymes. Treatment of the active form alone may allow for continued catabolic activity as the proenzymes are converted to active forms. The active form of the enzyme is treated by inhibiting activity, and the proenyme form is treated by preventing it from being activated.

Treatment of proenzyme and active proteinase enzymes includes, but is not limited to, the application of compounds that prevent the activation of proenzymes and inhibit the activity of proteinase enzymes. Compounds useful for treatment include, but are not limited to, small molecule compounds, antibodies, peptides, Tissue Inhibitors of Metalloproteinases (TIMPS), or other therapeutics known in the art. Many small molecule therapeutics are described for inhibiting MMPs that are involved in cancer metastasis. These can be efficaciously applied to the treatment of wounds via a variety of delivery vehicles and/or processes known to those skilled in the art in response to the results of the sensor. Correct dosing for treatment is achieved if the quantitative wound sensor is employed to determine the amount of enzyme that is present at the moment of testing. The sensor provides a way to increase the effectiveness of chronic wound treatment by temporally treating the current condition of the wound. The current state of the art would determine the status of the wound one or two days previous to reading the test results.

In another aspect of the invention, the wound sensor can be used to specifically identify the presence and type of any contaminating bacteria by using combinations of target and capture antibodies that detect bacterial cell surface markers. The bacterial wound sensor can be the same one that detects the proteinases by providing at least one reaction site for bacteria. Alternatively, a separate sensor can be provided to determine the presence of bacteria.

The following examples demonstrate the efficacy of the sensor and methods of the present invention.

### EXAMPLE 1

To test the sensor, human serum was spiked with proteinase enzymes to duplicate the testing of wound fluid. The proteinase enzymes MMP-1, MMP-8, MMP-9 and hNE were purchased from Calbiochem, Inc. Human serum was prepared from whole blood that had been collected in a heparinized vacutainer tube, by centrifuging at a speed of 3,000×g for 15 minutes and decanting the serum fraction. The proteinases were added to the serum to a final concentration of 50 ng/ml for each enzyme. A sample of 50 uL of the spiked serum was used for the test sample.

Capture and target antibodies to the proteinase enzymes MMP-1, MMP-8, MMP-9 and bNE were purchased from Sigma Chemicals, Inc. The anti-human MMP or hNE monoclonal antibodies (mAbs) were thiolated by dilution into phosphate buffered saline (PBS) (10 mM Kpi, pH 7.4, 150 mM NaCl) to a final concentration of 2 mg/mL. A fresh stock of 1.2 mM Sulfo-SPDP purchased from Pierce, was added to the antibody solution to a final concentration of 0.12 mM. The reaction mixture was stirred at room temperature for 60 minutes. The thiolated mAbs were purified via a 5 mL desalting column run in PBS. Fractions containing protein were pooled and concentrated to 10 mg/mL via Centricon (Amicon, Inc.). Capture antibodies were dotted onto a strip of nitrocellulose membrane and air-dried.

Target antibodies were affixed to carboxylated polystyrene beads of 0.3 micron diameter pre-labeled with a blue dye, purchased from Bangs Labs. For the coupling reaction, the beads were reacted with 50 mM N-hydroxysuccinimide, 0.2 M N-ethyl-N′-(dimethylaminopropyl)-carbodiimide in PBS at room temperature with slow mixing for 30 minutes. Next, 2-(2-pyridinylithio) ethaneamine (PDEA) was dissolved in 0.1 M borate buffer (pH 8.5) to a final concentration of 80 mM and added to the PM4 complex to a final concentration of 40 mM. The mixture was stirred at room temperature for one hour in the dark. Thiolated monoclonal antibody was added to the stirring mixture. Typically, 2 mg of mAb was used per coupling reaction. The mixture was incubated at room temperature for an hour with gentle stirring. Cystamine-HCl was added to the reaction at a final concentration of 40 mM. Incubation continued for an additional thirty minutes. The final complex was purified from un-reacted species by cross flow dialysis.

The beads were added to 200 μL of the protease enzyme/serum mixture. The mixture was incubated at room temperature for 10 minutes. A sample of 50 μL of this mixture was spotted onto the bottom of a piece of nitrocellulose containing reaction sites dotted with capture antibodies and the liquid was allowed to move across the surface. Excess liquid flowed off the upper end of the nitrocellulose filter into an absorbent pad. From top to bottom, the spots correspond to: ProMMP-1, MMP1, hNE, MMP-8, and MMP-9.

A negative control served to ensure that the test did not result in any false negatives. The last reaction site containing capture antibody was a negative control containing an antibody against cobra venom toxin that was purchased from Sigma Chemical, Inc. Cobra venom toxin antigen is normally not found in human serum.

The negative control reaction site remained without color, while the reaction sites containing capture antibodies to the enzymes became blue in color. FIG. 4 demonstrates the detection of antibodies to the proteinase enzymes and the formation of color at the reaction site.

### EXAMPLE 2

Alternatively, the antibodies were coupled to the carboxyl groups on the polystyrene bead by reacting with 50 mM N-hydroxysuccinimide, 0.2 M N-ethyl-N′-(dimethylaminopropyl)-carbodiimide in PBS at room temperature with slow mixing for 30 minutes. Next, 2-(2-pyridinyldithio) ethaneamine (PDEA) was dissolved in 0.1 M Borate buffer (pH 8.5) to a final concentration of 80 mM and added to the bead mix to a final concentration of 40 mM. This mixture was stirred at room temperature for one hour in the dark. Thiolated anti-human MMp (or hNE) antibody was added to the stirring bead mixture. Typically 2 mg of mAb was used per coupling reaction. The mixture was incubated at room temperature for an hour with gentle stirring. Cystamine-HCl was added to the reaction at a final concentration of 40 mM. Incubation continued for an additional thirty minutes. The final complex was purified from unreacted species by filtering the mixture through Whatman filter paper, washing with 10 mM Tris (pH 7.1), and collecting the modified polystyrene bead-antibody complex.

### EXAMPLE 3

An ELISA analysis was performed to demonstrate that it is possible to use an antibody that detects an epitope in the cleavage region to differentiate between proMMPs and activated MMPs. ELISA analysis was performed on polyclonal antibodies produced against an 11 mer peptide, GVPDLGRFQTF, that spans the activation cleavage region of MMP-9. One microgram of protein was mixed with human plasma and absorbed to the wells of a 96-well microtiter plate. The initial volume was 50 μL. After the wells were blocked with phosphate buffered saline (PBS) supplemented with 10% nonfat dry milk (blocking buffer), polyclonal antibodies in PBS were added at various dilutions and allowed to react with the antigen at room temperature for one hour. Following three washes in PBS, visualization was achieved via a goat anti rabbit secondary antibody that was conjugated with horseradish peroxidase. The secondary antibody was added at a 1:2000 dilution in blocking buffer and incubated at room temperature for one hour. After three washes in PBS, color development was achieved by adding a solution containing 50 mM sodium citrate, 50 mM citric acid, 1 mg/mL o-phenylenediamine, and 0.006% H₂O₂. After suitable color development, typically 5 to 10 minutes of incubation at room temperature, 50 μL of 2 M sulfuric acid was added to stop the reaction and stabilize the product. Absorbance was measured at 490 nm using an automatic ELISA plate reader.

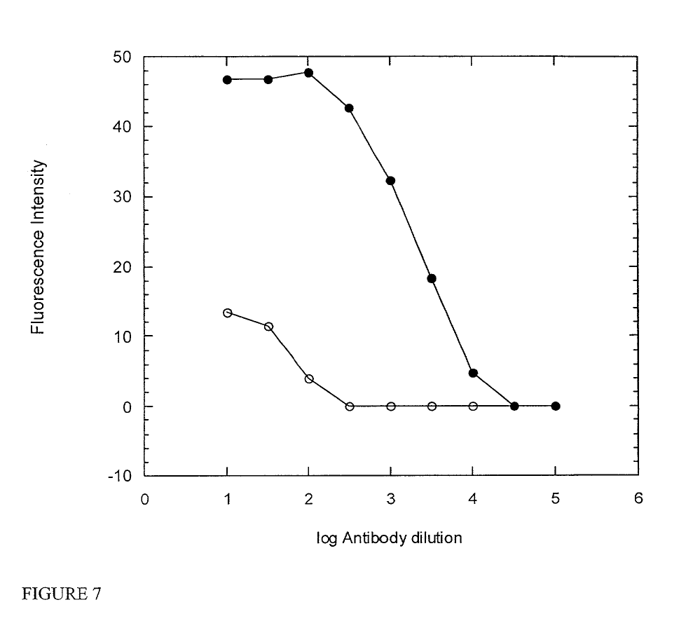

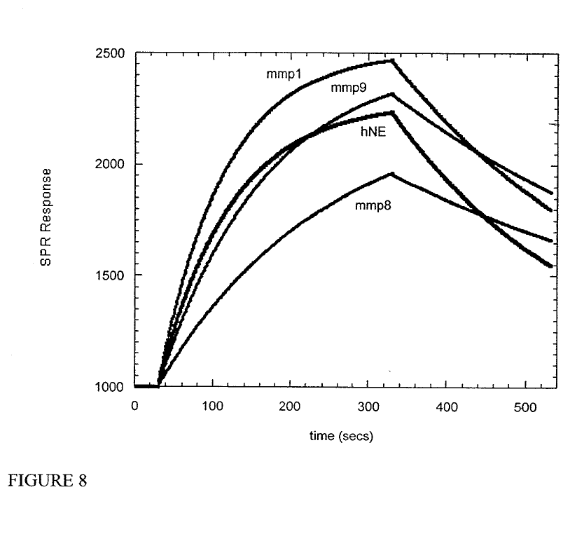

As shown in FIG. 7, the antibodies cross reacted with all three MMP forms, but preferentially cross react with the proMMP form. On the graph, the proMMP-9 is represented by closed circles and activated MMP-9 by open circles. The MMP activation region shows a high degree of primary sequence conservation. It is therefore expected that antibodies produced against this region will detect the proMMP form of most wound site MMPs. Although the observation that sequences downstream of the cleavage site show less conservation, it may mean that antibodies can be produced that are specific for individual MMPs.

### EXAMPLE 4

This example demonstrates the specific detection of the proenzyme form of wound site proteinases by polycolonal antibodies. The polyclonal antibodies produced here broaden the detection capabilities of the test and can be used directly in a variety of immunological formats including ELISA. They can also be modified prior to use for fluorescent or other assays. Hence, the polyclonal antibodies can be used in almost any clinical setting. An 11 amino acid peptide (GVPDLGRFQTF) that spans the cleavage site of MMP-9 was synthesized using standard peptide chemistries. Since small peptides do not illicit an immune response in animals, it was necessary to conjugate the peptide to a carrier protein. The peptide was conjugated to BSA using 1-ethyl-3-[3-dimethylaminopropyl]carbodiimide (EDC). This material was used to produce polyclonal antibodies (pAbs) in rabbits. The resulting antisera was purified using standard antibody purification techniques. The purified pAbs reacted preferentially with proMMP-9 demonstrating that they detect activated MMP-9. They can detect activated MMP-9 and the amino terminal proenzyme region, but to a much lesser extent. The detection of the various MMP forms can take place in simulated chronic wound fluid.

### EXAMPLE 5

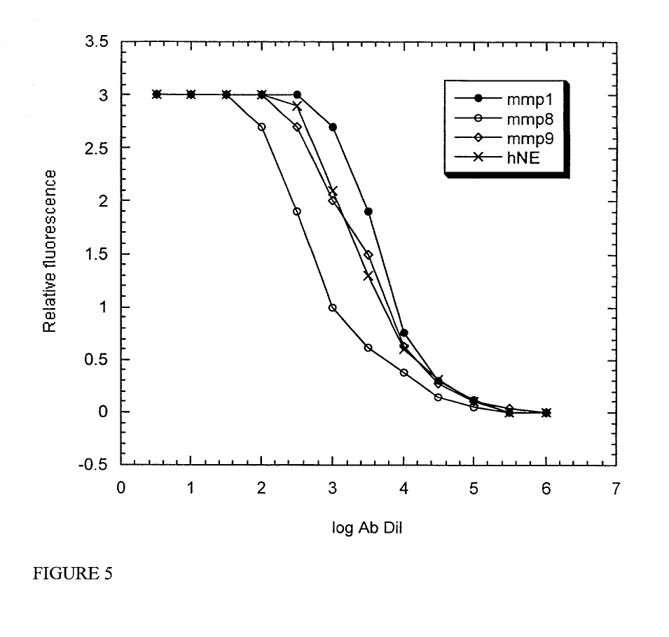

Testing was done to assure that the antibodies used in the sensor specifically recognize MMPs or hNE and were sufficiently sensitive to detect the enzymes at physiological concentrations. For the ELISA reaction, one microgram of human MMP or hNE was absorbed to the surface of a 96-well microtiter plate. The wells were then blocked with 10% non-fat dry milk in PBS. The blocked wells were washed three times with PBS. The antibodies were diluted into PBS to the same concentration (as monitored by A280) and serial dilutions were prepared in PBS. Aliquots of these dilutions were added into the microtiter wells and were allowed to react at room temperature for one hour. The wells were then washed three times with PBS. For the assay, a goat anti-mouse secondary antibody, conjugated to Pacific Blue (Molecular Probes, Inc.), was utilized for the detection phase of the ELISA assay. A 1:2000 dilution of the secondary antibody conjugate was added into the microtiter wells and was allowed to incubate at room temperature for one hour. These wells were then washed three times with PBS. The fluorescence emission intensity was measured using a Dynex, Inc. fluorescence microtiter plate reader employing a 410 nm and 460 nm bandpass filter set. The relative fluorescence was plotted versus the log of the antibody dilution. FIG. 5 is a graph demonstrating the relative fluorescence by ELISA analysis of the antibodies used in the assay. The results demonstrate that the antibodies are sufficiently sensitive, and that fluorescence detection is an effective means to detect binding of the antibodies.

### EXAMPLE 6

An ELISA assay was performed to determine the sensitivity of antibodies to detect enzyme levels in wound exudate. Human serum was spiked with a known amount of MMPs 1, 8, 9, or hNE. The assay was performed the same as in Example 5 with the exception that 50 uL of the spiked serum was adsorbed to the wells, and a 1:1000 dilution of the primary antibody was employed.

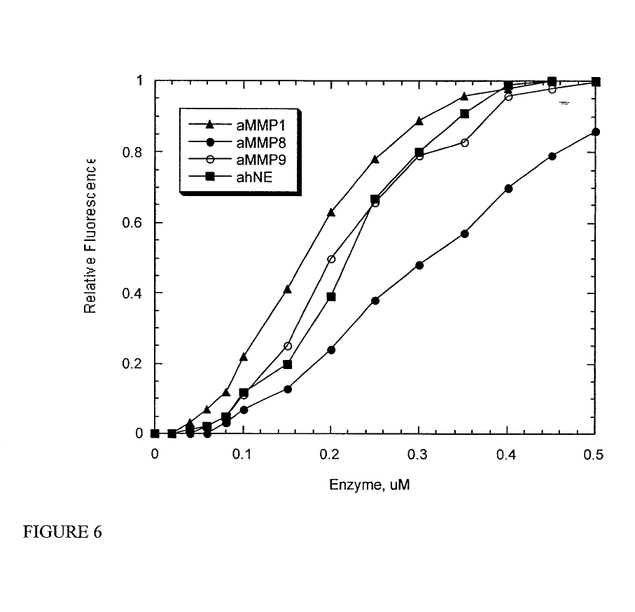

The results as shown in FIG. 6 demonstrate that about 0.1 uM of each enzyme was detected in serum. This is sufficient sensitivity to detect enzyme levels in wound exudate.

### EXAMPLE 7

Testing was performed to determine the amount of time necessary to form the antibody complexes in the sensor. The BiaCore-X surface plasmon resonance (SPR) device (BiaCore, Inc.) was utilized to measure the binding interactions. For these experiments, a carboxymethyl dextran sensor chip (CM-5) was activated with 50 mM N-hydroxysuccinimide, 0.2 M N-ethyl-N′-(dimethylaminopropyl)-carbodiimide at a flow rate of 10 uL per minute for 10 minutes. MMPs 1, 8, 9, or hNE at a concentration of 100 ng/ml each were then coupled to the activated surface at a flow rate of 10 uL per minute for ten minutes. The final surface was inactivated by flowing 1M ethanolamine-HCL at a rate of 10 uL per minute for five minutes over the sensor surface. Antibody, at a concentration of 100 ng/ml was then allowed to bind to the immobilized enzyme. All flow rates were at 10 uL/Min. FIG. 8 demonstrates the surface plasmon resonance detection of antibody binding. The graph demonstrates that antibody complex can be formed in 300 seconds.

Although the invention has been described in detail for the purpose of the illustration, it is understood that such detail is solely for that purpose, and variations can be made therein by those skilled in the art without departing from the spirit and scope of the invention, which is defined by the following claims.

We claim:

1. A sensor for detecting proteinase enzymes in a fluid comprising:
a) a sample reservoir having at least one target antibody and at least one signal element disposed therein, the target antibody bindable to a target proteinase enzyme upon exposure to the fluid to form a target proteinase enzyme target antibody complex; and
b) at least one reaction site in fluid communication with the sample reservoir and having a capture antibody bindable to the proteinase enzyme target antibody complex to form a target proteinase enzyme target antibody complex capture antibody conjugate, thereby indicating the presence of the target proteinase enzyme in the at least one reaction site by causing a detectable or measurable manifestation, wherein the target antibody is stationary within the reaction site.

2. The sensor of claim 1 further comprising a collection area in fluid communication with the at least one reaction site.

3. The sensor of claim 1 further comprising an absorbent pad.

4. The sensor of claim 3 wherein the absorbent pad is positioned within the sample reservoir, the collection area, or a combination thereof.

5. The sensor of claim 1 further comprising a wick disposed in and extending between the sample reservoir, the at least one reaction site and the collection area.

6. The sensor of claim 1 wherein the fluid communication is a channel, a capillary, a wick, or a combination thereof.

7. The sensor of claim 1, wherein the at least one reaction site comprises a plurality of reaction sites in liquid communication with the sample reservoir.

8. The sensor of claim 2, wherein the at least one reaction site comprises a plurality of reaction sites in liquid communication with the collection reservoir.

9. The sensor of claim 7, wherein each reaction site has a different capture antibody.

10. The sensor of claim 7, wherein the reaction sites are in fluid communication with each other.

11. The sensor of claim 1, further comprising at least one particle disposed within the sample reservoir and having at least one target antibody and at least one signal element attached.

12. The sensor of claim 11, wherein the particle is polymer, latex, gold, glass, silicon, metal, bacterial or fungal cell, or a combination thereof.

13. The sensor of claim 11, wherein the particle is a polystyrene bead.

14. The sensor or claim 1, wherein the signal element is a colorimetric compound, a radio-active compound, a potentiometric element, a fluorescent compound, a chemo-illuminescent compound, a light diffracting element, or a combination thereof.

15. The sensor of claim 1, further comprising a housing.

16. The sensor of claim 1, wherein the target proteinase enzyme is a proenzyme or an active enzyme.

17. The sensor of claim 1, wherein the target proteinase enzyme is MMP-1, MMP-8, MMP-9, hNE, pro MMP-1, proMMP-8, pro MMP-9, or combinations thereof.

18. A method for detecting the presence of at least one proteinase enzyme in a fluid of a human or an animal comprising:
a) providing a sample of the fluid of the human or the animal;
b) exposing the sample to a signal element and at least one target antibody, the at least one target antibody bindable to the at least one proteinase enzyme to form a proteinase enzyme/target antibody complex; and
c) exposing the proteinase enzyme/target antibody complex to form a proteinase enzyme/target antibody complex/capture antibody conjugate, to cause a detectable or measurable manifestation of the signal element, thereby indicating the presence of the at least one proteinase enzyme.

19. The method of claim 18, further comprising identifying the at least one proteinase enzyme by determining the presence or absence of a detectable or measurable manifestation of the signal element at a location of the proteinase enzyme/target antibody complex/capture antibody conjugate.

20. The method of claim 18, wherein the at least one target antibody and the signal element are attached to a particle.

21. The method of claim 18, wherein the at least one proteinase enzyme is a proenzyme or an active enzyme.

22. The method of claim 18, wherein the at least one proteinase enzyme is MMP-1, MMP-8, MMP-9, hNE, pro MMP-1, pro MMP-8, pro MMP-9, or combinations thereof.

23. The method of claim 18, wherein the sample is exposed to a plurality of target antibodies, each target antibody being bindable to a different proteinase enzyme; and wherein the presence of a plurality of proteinase enzymes is detected simultaneously.

24. The method of claim 18, wherein the signal element is a colorimetric compound, a radio-active compound, a potentiometric element, a fluorescent compound, a chemo-illuminescent compound, a light diffracting element, or a combination thereof.

25. The method of claim 18, wherein the sample of fluid is taken directly from a wound of the human or animal.

26. A method for treating chronic wounds in a human or an animal comprising:
a) providing a sample of the fluid of the human or the animal;
b) exposing the sample to a signal element and at least one target antibody, the at least one target antibody bindable to the at least one proteinase enzyme to form a proteinase enzyme/target antibody complex; and
c) exposing the proteinase enzyme/target antibody complex to form a proteinase enzyme/target antibody complex/capture antibody conjugate, to cause a detectable or measurable manifestation of the signal element, thereby indicating the presence of the at least one proteinase enzyme.
d) identifying the at least one proteinase enzyme by determining the presence or absence of a detectable or measurable manifestation of the signal element; and
e) selecting a treatment for the wound that is effective for treating the identified proteinase enzyme.

27. The method of claim 26, wherein the treatment for the wound that is effective for treating the identified proteinase enzyme comprises a proteinase enzyme inhibitor bindable specifically to the identified proteinase enzyme.

28. The method of claim 26, wherein the identified proteinase enzyme is a proenzyme, an active enzyme, or a combination thereof.

29. The method of claim 26, wherein the proteinase enzyme inhibitor is a proenzyme inhibitor, an active enzyme inhibitor, or a combination thereof.

30. The method of claim 26, wherein a plurality of proteinase enzymes are identified simultaneously and a plurality of proteinase enzymes are treated simultaneously.

31. A sensor for detecting proteinase enzymes in a fluid comprising:
a) a sample reservoir having at least one target antibody disposed therein, the target antibody bindable to a target proteinase enzyme upon exposure to the fluid to form a target proteinase enzyme target antibody complex; and
b) at least one reaction site in fluid communication with the sample reservoir and having a capture antibody bindable to the proteinase enzyme target antibody complex to form a target proteinase enzyme target antibody complex capture antibody conjugate, thereby indicating the presence of the target proteinase enzyme in the at least one reaction site by causing a detectable or measurable manifestation, wherein the target antibody is stationary within the reaction site.

32. The sensor of claim 31 further comprising a collection area in fluid communication with the at least one reaction site.

33. The sensor of claim 31 wherein the fluid communication is a channel, a capillary, a wick, or a combination thereof.

34. The sensor of claim 31, wherein the at least one reaction site comprises a plurality of reaction sites in liquid communication with the sample reservoir.

35. The sensor of claim 31, wherein the at least one reaction site comprises a plurality of reaction sites in liquid communication with the collection reservoir.

36. The sensor of claim 31, wherein the at least one reaction site comprises a plurality of reaction sites, wherein each reaction site has a different capture antibody.

37. The sensor of claim 31, wherein the target proteinase enzyme is a proenzyme or an active enzyme.

38. The sensor of claim 31, wherein the target proteinase enzyme is MMP-1, MMP-8, MMP-9, hNE, pro MMP-1, proMMP-8, pro MMP-9, or combinations thereof.

39. A method for detecting the presence of at least one proteinase enzyme in a fluid of a human or an animal comprising:
a) providing a sample of the fluid of the human or the animal;
b) exposing the sample to at least one target antibody, the at least one target antibody bindable to the at least one proteinase enzyme to form a proteinase enzyme/target antibody complex; and
c) exposing the proteinase enzyme/target antibody complex to form a proteinase enzyme/target antibody complex/capture antibody conjugate, to cause a detectable or measurable manifestation, thereby indicating the presence of the at least one proteinase enzyme.

40. The method of claim 39, wherein the at least one proteinase enzyme is a proenzyme, an active enzyme, or a combination thereof.

41. The method of claim 39, wherein the at least one proteinase enzyme is MMP-1, MMP-8, MMP-9, hNE, pro MMP-1, pro MMP-8, pro MMP-9, or combinations thereof.

42. The method of claim 39, wherein the sample is exposed to a plurality of target antibodies, each target antibody being bindable to a different proteinase enzyme; and wherein the presence of a plurality of proteinase enzymes is detected simultaneously.

43. A method for treating chronic wounds in a human or an animal comprising:
a) providing a sample of the fluid of the human or the animal;
b) exposing the sample to at least one target antibody, the at least one target antibody bindable to the at least one proteinase enzyme to form a proteinase enzyme/target antibody complex; and
c) exposing the proteinase enzyme/target antibody complex to form a proteinase enzyme/target antibody complex/capture antibody conjugate, to cause a detectable or measurable manifestation, thereby indicating the presence of the at least one proteinase enzyme.
d) identifying the at least one proteinase enzyme by determining the presence or absence of a detectable or measurable manifestation; and
e) selecting a treatment for the wound that is effective for treating the identified proteinase enzyme.

44. The method of claim 43, wherein the treatment for the wound that is effective for treating the identified proteinase enzyme comprises a proteinase enzyme inhibitor bindable specifically to the identified proteinase enzyme.

45. The method of claim 43, wherein the identified proteinase enzyme is a proenzyme, an active enzyme, or a combination thereof.
